(19) United States
(12) Patent Application Publication
(10) Pub. No.: US 2022/0291210 A1
(43) Pub. Date: Sep. 15, 2022

(54) ANALYSIS METHOD AND ANALYSIS DEVICE

(71) Applicant: Hitachi High-Tech Corporation, Tokyo (JP)

(72) Inventor: Hiroaki NAKAGAWA, Tokyo (JP)

(21) Appl. No.: 17/828,177

(22) Filed: May 31, 2022

Related U.S. Application Data
(62) Division of application No. 15/537,541, filed on Jun. 19, 2017, now abandoned.

(30) Foreign Application Priority Data
Dec. 29, 2014 (JP) 2014-267008

(51) Int. Cl. G01N 33/543; G01N 33/68; H01J 49/42

(52) U.S. Cl. CPC G01N 33/543; G01N 33/54306; G01N 33/6848; C12Y 302/01023; H01J 49/4225; G01N 2333/94; G01N 2333/924; G01N 2560/00

(57) ABSTRACT

Provided is a high-accuracy analysis method utilizing an enzyme-lined immunoassay. The presence of an analyte 3 can be detected or the abundance of the analyte 3 can be analyzed by: bonding an antibody 5 that is capable of specifically bonding to the analyte 3 immobilized on a solid phase 1 and has an enzyme 7 bonded thereto; then decomposing an enzyme substrate 8, which can generate decomposition products capable of being detected easily with a mass spectrometry, with the enzyme 7 bonded to the antibody 5; and then analyzing the decomposition products 9 and 10 with a mass spectrometry.

### TECHNICAL FIELD

The present invention relates to an analysis method and an analysis device, and particularly relates to an analysis technique using an enzyme-lined immunoassay.

### BACKGROUND ART

In recent years, an immunoassay utilizing antibody antigen reaction in which an antibody of biological defense protein is strongly bonded to a substance having a specific structure has been widely used for detection or quantitative analysis of biological components such as protein. In particular, an enzyme-lined immunoassay for measuring coloration or fluorescence of a product generated from an enzyme substrate by utilizing enzyme reaction, a multi-molecular enzyme reaction product is generated by the enzyme reaction. Accordingly, the enzyme-lined immunoassay has been widely used in order to relatively easily obtain sensitivity.

As an example of the enzyme-lined immunoassay, an analyte is bonded to an anti-analyte antibody bonded in a solid phase, and the anti-analyte antibody to which a label for specifically recognizing the analyte is bonded is bonded to the analyte. A complex not bonded to the analyte is removed, and a complex between a label bonded substance specifically bonded to the label and an enzyme is bonded to the label. Thereafter, the complex not bonded to the label is removed, and an enzyme substrate which reacts with the enzyme is added thereto, thereby generating an enzyme reaction product. In the related art, the enzyme reaction product whose spectroscopic properties such as absorbance or fluorescence are greatly changed by reaction or which further reacts with other substances is selected. In this manner, the presence or absence and concentration of the analyte are analyzed by analyzing a change in the absorbance or the fluorescence before and after the enzyme reaction.

The above-described configuration is just an example. Various methods have been developed in the enzyme-lined immunoassay. The enzyme-lined immunoassay also includes a method in which hydrophobicity or iconicity adsorption and chemical bonding are used in order to fix the analyte to a solid phase without using an antibody, and a method in which the enzyme is bonded to the anti-analyte antibody. As a type of the enzyme to be used, various enzymes such as β-galactosidase, peroxidase, alkaline phosphatase, acetylcholinesterase, and luciferase are commercially available for this purpose (refer to NPL 1).

On the other hand, a mass spectrometer (MS) is a device which ionizes a substance so as to measure m/z (value obtained in such a way that mass is divided by a charge number) and strength, based on mobility of the ion in vacuum. Although a macromolecule such as protein can be directly analyzed, sensitivity is degraded in many cases. The protein inside a living body receives post-translational modification such as alkylation, phosphorylation, and glycosylation, thereby causing diversity to occur in a molecular weight or a charge state. Accordingly, the protein is normally detected with different m/z. For these reasons, MS is less likely to directly measure the protein. Therefore, in order to measure the protein by using MS, the protein is cut into peptides by using trypsin, and the peptide suitable for MS detection is analyzed after being selected therefrom. In this manner, currently, the protein can be detected up to a level of several hundred pg (several fmol) (refer to NPL 2 and NPL 3).

### CITATION LIST

### Non-Patent Literature

NPL 1: The Immunoassay Handbook Fourth Edition, David Wild Edition, 2013, Elsevier Publisher (UK)
NPL 2: Tujin Shi et al., Proteomics Vol. 12, No. 8, Pages 1074 to 1092, 2012
NPL 3: Daniel C Liebler et al., Biochemistry Vol. 52, Pages 3797 to 3806, 2013

### SUMMARY OF INVENTION

### Technical Problem

The above-described enzyme-lined immunoassay in the related art utilizing colorimetry or fluorescence is likely to receive the influence of turbidity or bubbles of a sample solution, and in addition, a material of a usable container is restricted. On the other hand, although the above-described MS can generally perform high sensitivity analysis, the molecular weight or the ionization varies depending on substances. Consequently, analysis conditions or sensitivities greatly vary. In addition, due to the presence of other substances, a phenomenon called ion suppression occurs in which the ionization is hindered and the sensitivity is degraded. Therefore, in many cases, the sensitivity is degraded or data reproducibility is remarkably degraded. Conversely, in some cases, a phenomenon called ion enhancement also occurs in which the ionization is promoted. In particular, high performance liquid chromatography/MS (LC/MS) for a sample having many contaminants, such as biological samples and foodstuff, data reliability greatly depends on pretreatment or LC separation conditions. Therefore, in order to develop a new test item or to improve accuracy in a clinical test field, an analysis method for further improving the sensitivity is required.

An object of the present invention is to provide an analysis method and an analysis device which can improve sensitivity of an enzyme-lined immunoassay and can accurately analyze substances having various molecular weights, such as protein.

### Solution to Problem

In order to achieve the above-described object, according to the present invention, there is provided an analysis method for measuring an analyte. In the analysis method, an antibody to be specifically bonded to an analyte immobilized on a solid phase and having an enzyme bonded thereto is bonded to the analyte. Enzyme reaction is caused to occur between the enzyme bonded to the antibody and an enzyme substrate. Mass spectrometry is performed on an enzyme reaction product of the obtained enzyme substrate so as to detect the presence or absence of the analyte and to measure concentration of the analyte.

In addition, in order to achieve the above-described object, according to the present invention, there is provided an analysis device for measuring an analyte. The analysis device includes an enzyme reaction unit that bonds an antibody to be specifically bonded to an analyte immobilized on a solid phase and having an enzyme bonded thereto to the analyte so as to cause enzyme reaction to occur between the enzyme bonded to the antibody and an enzyme substrate, and a mass spectrometry unit that performs mass spectrometry on an enzyme reaction product of the obtained enzyme substrate. The analysis device measures the presence or absence and concentration of the analyte.

### Advantageous Effects of Invention

The present invention can provide a device which can very sensitively analyze an analyte by using an enzyme-lined immunoassay and which can easily analyze various target substances such as protein.

### BRIEF DESCRIPTION OF DRAWINGS

FIG. 1 is a schematic diagram for describing a principle of an analysis method using an enzyme-lined immunoassay according to the present invention.

FIG. 2 is a view illustrating a configuration example of an analysis device according to Embodiment 1.

FIG. 3 is a view illustrating an example of an operation flow of an analysis method according to Embodiment 1.

FIG. 4 is a view illustrating an example of an operation flow of an analysis method according to Embodiment 2.

### DESCRIPTION OF EMBODIMENTS

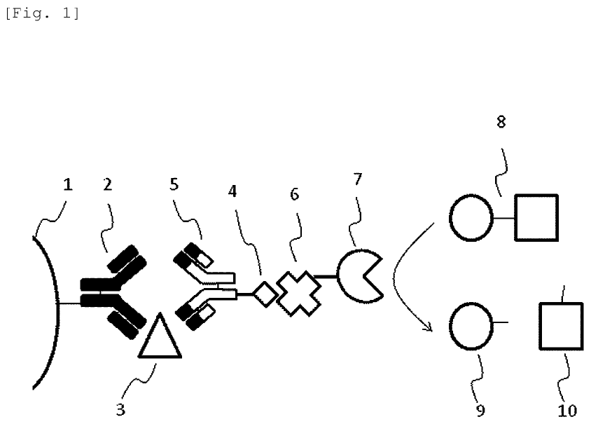

Hereinafter, preferred embodiments of an analysis method and an analysis device which utilize a new method in an enzyme-lined immunoassay according to the present invention will be described. First, a principle of the analysis method according to the present invention will be described with reference to an example of the enzyme-lined immunoassay illustrated in FIG. 1.

As illustrated in FIG. 1, an analyte 3 in an analysis sample is bonded to an anti-analyte antibody 2 bonded to a solid phase 1 such as a fine particle and a container surface. Thereafter, cleaning is performed so as to remove contaminants in the analysis sample. Next, an anti-analyte antibody 5 having a label 4 bonded thereto in order to specifically recognize the analyte 3 is bonded to the immobilized analyte 3 by antigen-antibody reaction. Thereafter, cleaning is performed so as to remove a complex of the label 4 not bonded to the analyte 3 and the anti-analyte antibody 5. Subsequently, a complex of a label bonded substance 6 to be specifically bonded to the label 4 and an enzyme 7 is added so as to bond the label 4 and the label bonded substance 6 to each other. Thereafter, cleaning is performed so as to remove a complex of the label bonded substance 6 not bonded to the label 4 and the enzyme 7.

Furthermore, if an enzyme substrate 8 to react with the enzyme 7 is added, the enzyme substrate 8 generates enzyme reaction products 9 and 10 through enzyme reaction. As an example, the label 4 and the label bonded substance 6 include a combination of biotin and avidin or streptavidin. However, the label bonded substance 6 may be an anti-labeled antibody by setting the label 4 to any optional compound. Without using the label 4, an anti-anti-analyte antibody for recognizing the anti-analyte antibody 5 or antibody bonding protein such as protein G or A may be used.

A specific example of the enzyme reaction caused by the enzyme substrate is as follows. In the reaction of β-galactosidase, p-nitrophenyl β-D-galactoside is decomposed, and p-nitrophenol is liberated. This p-nitrophenol is colored in weak alkaline, thereby enabling this p-nitrophenol to be measured by being absorbed as much as 420 nm.

In peroxidase, hydrogen peroxide is decomposed, 3,3′, 5,5′-tetramethylbenzidine is oxidized by active oxygen generated in response thereto, is colored in acid, and is measured by being absorbed as much as 450 nm. Alkaline phosphatase decomposes p-nitrophenyl phosphate, and p-nitrophenol is formed, thereby measuring the absorbance at 405 nm. Acetylcholinesterase decomposes acetylcholine into acetic acid and thiocholine. This thiocholine cleaves disulfide bonding of 5,5′-dithio-bis-(2-nitrobenzoic acid), and 5-thio-2-nitrobenzoic acid is formed, thereby measuring the absorbance at 412 nm.

In addition to this configuration, as a substrate of β-galactosidase, phenyl β-D-galactoside, p-aminophenyl β-D-galactoside, p-methoxyphenyl β-D-galactoside, o-nitrophenyl β-D-galactoside, p-methylumbelliferyl-β-galactoside, p-5-bromo-4-chloro-3-indolyl β-D-galactopyranoside, 5-bromo-6-chloro-3-indolyl β-D-galactopyranoside, 5-bromo-3-indolyl β-D-galactopyranoside, 6-chloro-3-indolyl β-D-galactopyranoside, and o-nitrophenyl β-D-galactopyranoside are known.

As a substrate of peroxidase, 4-aminoantipyrine, 2,2′-azinobis (3-ethylbenzothiazoline-6-ammonium sulfonate), 5-aminosalicylic acid, 3,5-dichloro-2-hydroxybenzenesulfonic acid, 2,4-dichlorophenol, N,N-dimethylaniline, 3-diethylaminotoluene, 3-methyl-2-benzothiazolinone hydrazone, 2,2′-azinodi (3-ethylbenzthiazolidine)-6′-sulfonate, 1,2-phenylenediamine, 3-(3,5-dimethoxyanilino)-2-hydroxypropanesulfonic acid, 2,4,6-tribromo-3-hydroxybenzoic acid, tyramine, and p-hydroxyphenylpropionic acid are known. As a substrate of alkaline phosphatase, 4-methylumbelliferyl phosphate is known.

However, the enzyme-lined immunoassay in the related art utilizes colorimetry or fluorescence as described above. The enzyme-lined immunoassay is likely to receive the influence of turbidity or bubbles of a sample solution, and in addition, a material of a usable container is restricted.

Therefore, in the analysis method and the analysis device which utilize the enzyme-lined immunoassay according to the present invention, as the enzyme substrate reacting with the enzyme, a compound whose enzyme reaction product is likely to be detected through mass spectrometry using the mass spectrometer (MS). Although MS has various types, it is more preferable to use an electrospray ionization-triple quadrupole MS. The electrospray ionization is a method in which a substance in a solution is stably ionized by a potential difference and spray gas. Three electrodes of the quadrupole MS are arranged in series, and ion of any optional m/z is selected by the first quadrupole. The ion is decomposed by the subsequent quadrupole, and any optional decomposed ion is detected by the last quadrupole. Due to a difference in m/z, most other substances are separated by the first quadrupole. A decomposition pattern is characterized by a compound in the subsequent quadrupole. Accordingly, even in a case of the compound accidentally mixed with the same m/z in the first quadrupole, a deposition product varies. It is possible to very selectively measure the amount of a specific substance in the last quadrupole.

The compound which is likely to be detected by MS having this configuration is likely to be ionized by the electrospray, and is likely to be quantified by the triple quadrupole. The enzyme reaction product 9 which serves as a major analyte illustrated in FIG. 1 reduces the influence of ion suppression of the enzyme reaction product 10 which does not serve as the enzyme substrate 8 or the other major analyte. Accordingly, it is desirable to separate these components by using liquid chromatography (LC). When the compound is ionized by the electrospray, the compound having higher organic solvent concentration is likely to be vaporized, and ionization efficiency is satisfactory. As the compound whose organic solvent concentration becomes higher in the column of C18 system widely used in LC, it is preferable to select the compound whose log P value is 1 to 5. In addition, an organic compound in which a heteroatom is contained in nitrogen or oxygen carbon, and oxygen or nitrogen other than hydrogen is likely to be ionized.

A second characteristic to facilitate analysis using MS is that interference between compounds does not occur. In some cases, interference occurs in the presence of an isotope element or in the mass spectrometry using an adduct ion, a dehydration ion, or the like even though masses of compounds are different. In order to eliminate this case, it is desirable that difference between m/z values is by 40 or more. In addition, if compounds are simultaneously ionized even though the compounds have different masses, there is a high possibility of causing interference called ion suppression. In the enzyme reaction, a reaction rate is increased by increasing the concentration of a reaction substrate, and thus, it is expected that the concentration of the reaction substrate becomes higher than that of a detected compound and the ionization of the detected compound is inhibited.

In the enzyme reaction system of the analysis method using the enzyme-lined immunoassay according to the present invention, the enzyme is immobilized into the solid phase, and thus, the enzyme substrate and salts or organic solvents such as a reaction product or a buffer solution are present. In order to easily separate the detected compound from the enzyme substrate through chromatography, it is desired that chemical properties are significantly different. For example, in a case where β-galactosidase which is one of glycolytic enzymes is used as the enzyme and p-nitrophenyl-β-galactoside is used as the enzyme substrate, the reaction product is p-nitrophenol used in detection and galactose recognized by the enzyme. These components are easily separated through hydrophobic chromatography such as C18 having significantly different polarities. In addition, examples of impurities in MS include water or an organic solvent such as water or acetonitrile, used as a mobile phase, and a buffer solution component such as ammonia or formic acid or a cluster formed by gathering several molecules thereof, in addition to compounds derived from the sample. To avoid generation of the impurities, it is desired that m/z is 150 or greater. However, if the mass is increased, it cannot be interpreted that polyvalent ions are easily generated, and thus, it is preferable that the molecular weight of the compound is 1,000 or less.

In view of the above-described configuration, in the analysis method using the enzyme-lined immunoassay according to the present invention, it is desired that an enzyme reaction product which is a MS analysis object generated by the enzyme reaction has log P, which is an index of hydrophobicity, of 1 to 5, and is a compound having a molecular weight of 150 to 1,000. Furthermore, in the triple quadrupole MS, compound is decomposed in a second triple quadrupole, and analysis accuracy is improved using the fact decomposition patterns thereof are different for each compound. In some cases, it is less likely to be interpreted that decomposition does not easily occur in a small molecule and decomposition is too complicated in a large molecule. Stable decomposition easily occurs in a structure in which a plurality of aromatic compounds are connected via a linker including a single bond of C—N or C—O. In the light of this, in the analysis method and the analysis device according to the present invention, it is desired that the enzyme reaction product which is a MS analysis object has a structure in which a plurality of aromatic compounds are connected via a linker including a single bond of C—N or C—O, and is a compound having a molecular weight of 200 to 600. As an example of the enzyme reaction product, verapamil (C27H₃₈O₄, molecular weight of 454.61) is used.

### Embodiment 1

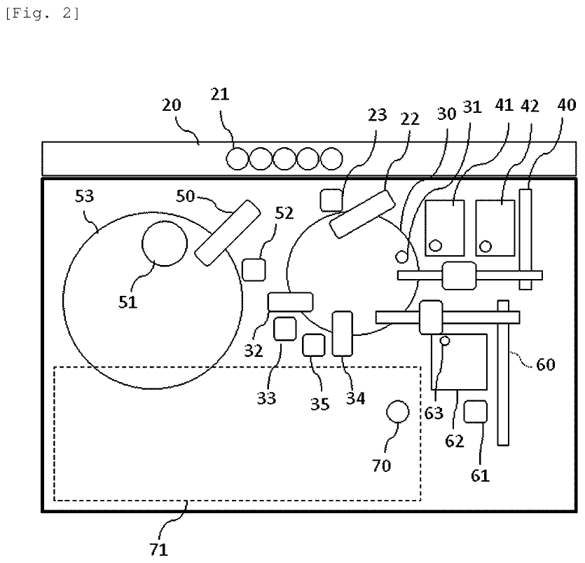

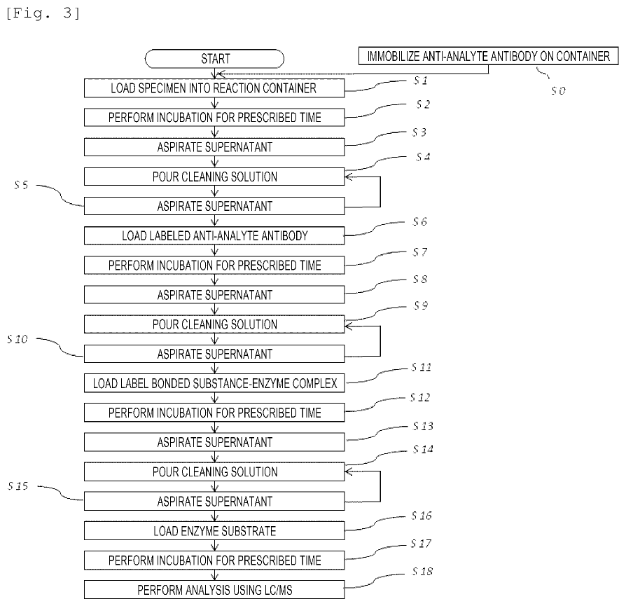

Subsequently, a first embodiment of the analysis method and the analysis device according to the present invention will be described with reference to FIG. 2 and FIG. 3.

The present embodiment is an embodiment of an analysis method in which an antibody to be specifically bonded to an analyte immobilized on a solid phase and having an enzyme bonded thereto is bonded to the analyte, enzyme reaction is caused to occur between the enzyme bonded to the antibody and an enzyme substrate, and mass spectrometry is performed on an enzyme reaction product of the obtained enzyme substrate so as to detect the presence or absence of the analyte and to measure concentration of the analyte. In addition, the present embodiment is an embodiment of the analysis device which includes an enzyme reaction unit that bonds an antibody to be specifically bonded to an analyte immobilized on a solid phase and having an enzyme bonded thereto to the analyte so as to cause enzyme reaction to occur between the enzyme bonded to the antibody and an enzyme substrate, and a mass spectrometry unit that performs mass spectrometry on an enzyme reaction product of the obtained enzyme substrate, and detects the presence or absence of the analyte and measures concentration of the analyte.

The automatic analysis device according to Embodiment 1 shown in FIG. 2 includes a mass spectrometry unit configured of a LC/MS 71 indicated by dotted lines and other constituent elements, but in the present specification, constituent elements other than the mass spectrometry unit are collectively referred to as an enzyme reaction unit. In the enzyme reaction unit, an antibody to be specifically bonded to an analyte immobilized on a solid phase and having an enzyme bonded thereto is bonded to the analyte, and enzyme reaction is caused to occur between the enzyme bonded to the antibody and the enzyme substrate.

As illustrated in FIG. 2, in the enzyme reaction unit, a certain amount of a specimen such as blood serum or a standard solution is transferred from a specimen container 21 in a specimen supply unit 20 into a reaction container 31 on a reaction table 30 using a specimen dispensing device 22 (S1 in the operation flow in FIG. 3, the same as above hereinafter). The specimen dispensing device 22 transferred to the reaction container 31 is cleaned using the nozzle cleaning mechanism 23. In the outer circumference of the reaction table 30, a plurality of reaction containers can be held, and the reaction container 31 can be moved to an arbitrary operation part by a rotary motion of the reaction table 30. In addition, a constant temperature function, a stirring function, a magnetic collection function, or the like can be appropriately added to the reaction table 30. The reaction container 31 on the reaction table 30 is supplied from a reaction container stocker 41 by the action of a reaction container transfer unit 40. In each reaction container, a specific anti-analyte antibody 2 is bonded to and immobilized on an analyte 3 in advance (S0).

The reaction container 31 on the reaction table 30 containing the specimen is transferred to an operation part of a suction nozzle 32 after a prescribed time (S2), and a supernatant after reaction is sucked and removed (S3). The suction nozzle 32 is cleaned using the nozzle cleaning mechanism 33. Next, the reaction container 31 is transferred to an operation part of a cleaning solution supply nozzle 34, and a cleaning solution is charged (S4). The cleaning solution supply nozzle 34 is cleaned using a nozzle cleaning mechanism 35. The reaction container 31 is cleaned several times by reciprocating between the suction nozzle operation part and the cleaning solution supply nozzle operation part (S4 and S5).

The cleaned reaction container 31 is transferred to an operation part of a reagent supply nozzle 50, and filled with a complex of a label 4 and the anti-analyte antibody 5 from a reagent container 51 (S6). The reagent supply nozzle 50 is cleaned using a nozzle cleaning mechanism 52. A reagent table 53 has a plurality of reagent containers 51 stored therein, and has an appropriate constant temperature function or stirring function. The reaction container 31 is transferred to the operation part of a suction nozzle 32 after a certain time (S7), and a supernatant after reaction is sucked and removed (S8). The reaction container 31 is cleaned several times by reciprocating between the suction nozzle operation part and the cleaning solution supply nozzle operation part (S9 and S10).

Next, the reaction container 31 is transferred to the operation part of a reagent supply nozzle 50, and is filled with a complex of a label bonded substance 6 and an enzyme 7 (S11). The reaction container 31 is transferred to the operation part of a suction nozzle 32 after a certain time (S12), and a supernatant after reaction is sucked and removed (S13). The reaction container 31 is cleaned several times by reciprocating between the suction nozzle operation part and the cleaning solution supply nozzle operation part (S14 and S15).

Then, the reaction container 31 is transferred to the operation part of a reagent supply nozzle 50, and is filled with an enzyme substrate 8 (S16). The reaction container 31 is transferred to an operation part of a sample injection nozzle mechanism 60 after a certain time (S17), a certain amount of a specimen passes through an LC/MS sample injection unit 70, and analysis is performed in LC/MS 71 which is a mass spectrometry unit (S18). The sample injection nozzle mechanism 60 is cleaned using a cleaning mechanism 61. In a case where analysis cannot be immediately performed after the lapse of a certain time from filling with enzyme substrate 8, the reaction container 31 is filled with an enzyme reaction stop solution by the reagent supply nozzle 50, temporarily held in an analysis sample container 63 on an analysis sample table by the sample injection nozzle mechanism 60, and sequentially analyzed in LC/MS 71 configuring a mass spectrometry unit. The reaction container 31 into which the sample is completed injected is transferred to a completed reaction container accommodation unit 42 by the reaction container transfer unit 40.

According to the automatic analysis device and the analysis method of Embodiment 1 described above, it is possible to very sensitively analyze an analysis target by the enzyme-lined immunoassay, and easily perform analysis of versatile substances such as protein using MS.

### Embodiment 2

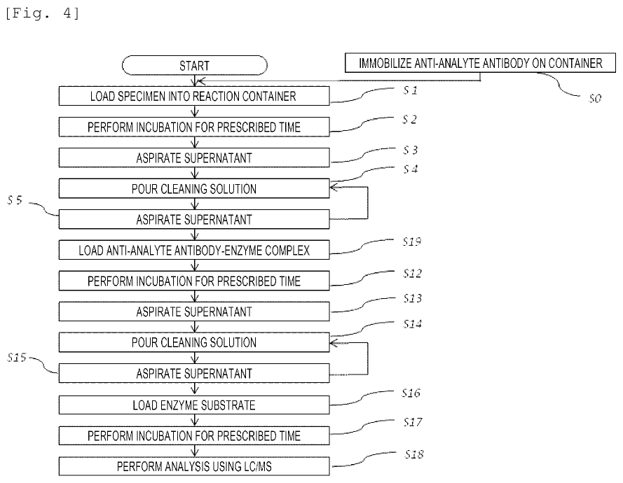

As described above, various methods are used for the enzyme-lined immunoassay, and as Embodiment 2, unlike the analysis method of Embodiment 1, an embodiment of using an anti-anti-analyte antibody for recognizing an anti-analyte antibody without using a label will be described. FIG. 4 shows an operation flow of the analysis method of Embodiment 2. In the operation flow shown in FIG. 4, the same reference signs as those in FIG. 3 denote the same operation, and the present operation flow can also be realized using the automatic analysis device shown in FIG. 2.

As illustrated in the same drawing, the reaction container 31 subjecting to cleaning several times (S4 and S5) is transferred to the operation part of the reagent supply nozzle 50, and in the present embodiment, the reaction container 31 is filled with a complex of the anti-analyte antibody 5 and the enzyme 7 (S19). Next, similar to Embodiment 1, the reaction container 31 is transferred to the operation part of the suction nozzle 32 after a certain time (S12), and a supernatant after reaction is sucked and removed (S13). The reaction container 31 is cleaned several times by reciprocating between the suction nozzle operation part and the cleaning solution supply nozzle operation part (S14 and S15). Then, the reaction container 31 is transferred to the operation part of the reagent supply nozzle 50 and is filled with the enzyme substrate 8, and the enzyme reaction is performed (S16). The reaction container 31 is transferred to the operation part of the sample injection nozzle mechanism 60 after a certain time (S17), a certain amount of a specimen passes through the LC/MS sample injection unit 70, and analysis is performed in LC/MS 71 which is a mass spectrometry unit (S18).

According to the present embodiment, the label is not used, and thus, it is possible to simplify analysis procedures. In addition, similar to Embodiment 1, it is possible to very sensitively analyze an analysis target by the enzyme-lined immunoassay, and easily perform analysis of versatile substances such as protein using MS.

Subsequently, analysis examples in which analysis is performed by applying various embodiments described above will be sequentially described.

### Analysis Example 1

Prior to Analysis Examples 2 to 5, as Analysis Example 1, galactosidase which is the enzyme 7 was caused to react respectively with p-nitrophenyl-β-galactoside, 4-methylumbelliferyl-β-galactoside, and a verapamil derivative of β-galactoside, as the enzyme substrate 8, and reaction products were analyzed respectively by LC/MS which is a the mass spectrometry unit and the spectrometric analysis device. 4-Nitrophenol (C₆H₅NO₃, molecular weight of 139.11) which is one of the enzyme reaction products can be obtained with detection sensitivity similar to spectra in LC/MS. 4-Methylumbelliferone (C₁₀H₈O₃, molecular weight of 176.171) which is one of the reaction products can be detected with higher sensitivity than spectra in LC/MS. Furthermore, verapamil (C27H₃₈O₄, molecular weight of 454.61) which is one of the reaction products can be detected with extremely high sensitivity in LC/MS.

### Analysis Example 2

The present analysis example is an analysis example related to Embodiment 1 described with reference to FIG. 2. Human serum albumin, as the analyte 3 which is a specimen, was dissolved in a buffer solution in a concentration range of 50 pg/mL to 20 ng/mL, and 50 μL of the resulting product was dispensed into the reaction container 31 coated with an anti-human serum albumin antibody which is the anti-analyte antibody 2. Then, 100 μL of a biotin labeled anti-human serum albumin antibody solution which is a complex of the anti-analyte antibody 5 and the label 4 was put into the reaction container 31, and the reaction container was incubated for one hour. A supernatant was sucked, and the reaction container 31 was cleaned three times with tris-buffered saline. 100 μL of a streptavidin galactosidase complex solution as a complex of the label bonded substance 6 and the enzyme 7 was added into the reaction container 31, the container was incubated for one hour. Then, a supernatant was sucked, and the container was cleaned three times with tris-buffered saline. p-Nitrophenyl-β-galactoside and 4-methylumbelliferyl-β-galactoside, as the enzyme substrate 8, were reacted respectively therein and reaction products were analyzed respectively by LC/MS and the spectrometric analysis device.

### Analysis Example 3

The present analysis example is an analysis example related to Embodiment 1 described with reference to FIG. 2. Human serum albumin, as the analyte 3 which is a specimen, was dissolved in a buffer solution in a concentration range of 50 pg/mL to 20 ng/mL, and 50 μL of the resulting product was dispensed into the reaction container 31 coated with an anti-human serum albumin antibody which is the anti-analyte antibody 2. 100 μL of a biotin labeled anti-human serum albumin antibody solution which is a complex of the label 4 and the anti-analyte antibody 5 was put therein, and the reaction container was incubated for one hour. Then, a supernatant was sucked, and the reaction container was cleaned three times with tris-buffered saline. 100 μL/well of a streptavidin galactosidase complex solution which is a complex of the label bonded substance 6 and the enzyme 7 was added into the reaction container 31, the reaction container was incubated for one hour. Then, a supernatant was sucked, and the reaction container was cleaned three times with tris-buffered saline. A verapamil derivative of β-galactoside which is the enzyme substrate 8 was reacted therein and reaction products were analyzed respectively by high performance liquid LC/MS.

### Analysis Example 4

The present analysis example is an analysis example related to Embodiment 1 described with reference to FIG. 2. Human C-reactive protein, as the analyte 3 which is a specimen, was dissolved in a buffer solution in a concentration range of 50 pg/mL to 16 ng/mL, 50 μL of the resulting product was dispensed into the reaction container 31 coated with an anti-human C-reactive protein antibody which is the anti-analyte antibody 2, and the reaction container was incubated for two hours. Then, a supernatant was sucked, and the reaction container was cleaned five times with 200 μL of a cleaning solution. 50 μL of a biotinylated anti-human C-reactive protein antibody as a complex of the label 4 and the anti-analyte antibody 5 was added thereto, and the reaction container was incubated for 30 minutes. A supernatant was sucked, and the reaction container was cleaned five times with 200 μL of a cleaning solution. 50 μL of a streptavidin galactosidase complex solution which is a complex of the label bonded substance 6 and the enzyme 7 was added into the reaction container 31, and the reaction container was incubated for 30 minutes. Then, a supernatant was sucked, and the reaction container was cleaned five times with 200 μL of a cleaning solution. p-Nitrophenyl-β-galactoside, 4-methylumbelliferyl-β-galactoside, and a verapamil derivative of β-galactoside which are the enzyme substrate 8 were reacted respectively therein, and reaction products were analyzed respectively by high performance liquid LC/MS and the spectrometric analysis device.

### Analysis Example 5

The present analysis example is an analysis example related to Embodiment 2 described with reference to FIG. 3. Rabbit IgG, as the analyte 3 which is a specimen, was dissolved in a buffer solution in a concentration range of 2 pg/mL to 100 ng/mL, magnetic particles, as a solid phase 1 coated with an anti-rabbit IgG antibody which is the anti-analyte antibody 2, were suspended in 100 μL of the solution, and the resulting product was incubated for one hour. Magnetic particles were collected by a magnet, a supernatant was sucked, and then the particles were cleaned five times with 200 μL of a cleaning solution. Next, 100 μL of galactosidase-conjugated anti-rabbit IgG antibody, as a complex of the anti-analyte antibody 5 and the enzyme 7, was added thereto, and the resulting product was incubated for one hour. Particles were collected by a magnet, and then were cleaned five times with 200 μL of a cleaning solution. p-Nitrophenyl-β-galactoside, 4-methylumbelliferyl-β-galactoside, and a verapamil derivative of β-galactoside which are the enzyme substrate 8 were reacted respectively therein, and reaction products were analyzed respectively by LC/MS and the spectrometric analysis device.

According to the analysis method and the analysis device of the present invention which are described above, clinical test items are widely expanded and accuracy in analyzing the clinical test items is improved by very sensitively analyzing biological components.

Furthermore, the present invention is not limited to the embodiments described above, and includes various modification examples. For example, the embodiments described above have been described in detail for facilitating better understanding of the present invention. A configuration of a certain embodiment can be partially substituted with a configuration of the other embodiment. In addition, a configuration of the other embodiments can be added to the configuration of the certain embodiment. In addition, with regard to a part of a configuration of each embodiment, additions, omissions, and substitutions of other configurations can be made.

### REFERENCE SIGNS LIST

1: SOLID PHASE,
2: ANTI-ANALYTE ANTIBODY,
3: ANALYTE,
4: LABEL,
5: ANTI-ANALYTE ANTIBODY,
6: LABEL BONDED SUBSTANCE,
7: ENZYME,
8: ENZYME SUBSTRATE,
9: ENZYME REACTION PRODUCT WHICH SERVES AS MAJOR ANALYSIS TARGET,
10: ENZYME REACTION PRODUCT WHICH DOES NOT SERVE AS MAJOR ANALYSIS TARGET,
20: SPECIMEN SUPPLY UNIT,
21: SPECIMEN CONTAINER,
22: SPECIMEN DISPENSING DEVICE,
23: NOZZLE CLEANING MECHANISM,
30: REACTION TABLE,
31: REACTION CONTAINER,
32: SUCTION NOZZLE,
33: NOZZLE CLEANING MECHANISM,
34: CLEANING SOLUTION SUPPLY NOZZLE,
35: NOZZLE CLEANING MECHANISM,
40: REACTION CONTAINER TRANSFER UNIT,
41: REACTION CONTAINER STOCKER,
42: COMPLETED REACTION CONTAINER ACCOMMODATION UNIT,
50: REAGENT SUPPLY NOZZLE,
51: REAGENT CONTAINER,
52: NOZZLE CLEANING MECHANISM,
53: REAGENT TABLE,
60: SAMPLE INJECTION NOZZLE MECHANISM,
61: NOZZLE CLEANING MECHANISM,
62: ANALYSIS SAMPLE TABLE,
63: ANALYSIS SAMPLE CONTAINER,
70: LC/MS SAMPLE INJECTION UNIT,
71: LC/MS

1. An analysis method for measuring an analyte,
wherein an analyte is immobilized on a solid phase,
wherein an antibody to which a label to be specifically bonded to the analyte immobilized on the solid phase is bonded to the analyte,
wherein a label bonded substance having galactosidase bonded thereto is bonded to the label,
wherein enzyme reaction is caused to occur between the galactosidase bonded to the label bonded substance and an enzyme substrate, and
wherein mass spectrometry is performed on an enzyme reaction product of the obtained enzyme substrate so as to measure the presence or absence and concentration of the analyte.

2. The analysis method according to claim 1,
wherein as the label and the label bonded substance, biotin and avidin are used.

3. The analysis method according to claim 1, comprising:
a step of immobilizing the analyte on the solid phase;
a step of bonding the antibody to be specifically bonded to the analyte and having the galactosidase bonded thereto to the analyte;
an enzyme reaction step of adding the enzyme substrate and causing the enzyme substrate to react with the galactosidase for a prescribed time; and
an analysis step of causing a mass spectrometer to analyze the obtained enzyme reaction product.

4. The analysis method according to claim 1,
wherein the enzyme reaction product serving as an analysis target is a compound in which log P serving as a hydrophobicity index is 1 to 5 and a molecular weight is 150 to 1,000.

5. The analysis method according to claim 4,
wherein the enzyme reaction product has a structure in which a plurality of aromatic compounds are linked by a linker including single bonding of C—N or C—O, and is a compound having the molecular weight of 200 to 600.
